(19) United States
(12) Patent Application Publication
(10) Pub. No.: US 2021/0161918 A1
(43) Pub. Date: Jun. 3, 2021

(54) TIGECYCLINE FOR TOPICAL TREATMENT OF ROOT CANAL SPACE

(71) Applicant: THE UNIVERSITY OF NORTH CAROLINA AT CHAPEL HILL, Chapel Hill, NC (US)

(72) Inventor: Ashraf F. Fouad, Chapel Hill, NC (US)

(21) Appl. No.: 16/957,497

(22) PCT Filed: Jan. 8, 2019
(86) PCT No.: PCT/US2019/012628

§ 371 Date: Jun. 24, 2020

Related U.S. Application Data
(60) Provisional application No. 62/615,561, filed on Jan. 10, 2018.

(51) Int. Cl. A61K 31/65; A61K 31/155; A61K 33/08; A61K 6/52; A61C 5/50; A61K 6/17; A61K 45/06; A61P 31/04

(52) U.S. Cl. CPC A61K 31/65; A61K 31/155; A61K 33/08; A61P 31/04; A61C 5/50; A61K 6/17; A61K 45/06; A61K 6/52

(57) ABSTRACT

The invention relates to methods for treating a bacterial infection in a root canal space of a tooth using topical application of tigecycline. The invention further relates to methods for reducing the risk of developing an infection in a root canal space of a tooth using topical application of tigecycline.

### PRIORITY

This application claims the benefit, under 35 U.S.C. § 119 (e), of U.S. Provisional Application No. 62/615,561 filed on Jan. 10, 2018, the entire contents of which is incorporated by reference herein.

### STATEMENT OF GOVERNMENT SUPPORT

This invention was made with government support under Grant Number DE015320 awarded by the National Institutes of Health. The government has certain rights in the invention.

### FIELD OF THE INVENTION

The invention relates to methods for treating a bacterial infection in a root canal space of a tooth using topical application of tigecycline. The invention further relates to methods for reducing the risk of developing an infection in a root canal space of a tooth using topical application of tigecycline as the sole active agent or in a combinatorial formulation.

### BACKGROUND OF THE INVENTION

Endodontic infections are polymicrobial infections that may result in significant morbidity, tooth loss, hospital stay or even mortality. The control of these infections is by endodontic treatment or tooth extraction. The effectiveness of root canal treatment or retreatment in cases with pre-existing infection is significantly reduced compared to cases with no infections. This healing of established infections may take 1-4 years to be seen radiographically, and occurs in about 78-85% of adequately-treated cases, whereas the tooth socket heals within weeks radiographically if the tooth were to be extracted. Currently, there are no predictable methods for increasing the healing rates of infection in teeth treated endodontically, or accelerating the healing of established infection after root canal treatment. Disinfection of the root canal space has been shown to play an important role in the success of regenerative endodontic procedures (1-3). The commonly accepted disinfection protocol for regenerative endodontic procedures is irrigation of the root canal space with sodium hypochlorite (NaOCl) in the first visit and then dressing the root canal with triple antibiotic paste (TAP) (4-6), which includes equal proportions of ciprofloxacin, metronidazole, and minocycline (7). However, while TAP is effective at concentrations of 1 mg/mL, the minocycline causes severe discoloration of the tooth. In addition, at this concentration, TAP leads to cytotoxic effects by killing the stem cells within the canal and may prevent revascularization within the pulp region, thereby inhibiting pulp tissue regeneration. While the beta lactam antibiotics such as amoxicillin and AUGMENTIN® were shown to be most effective against endodontic pathogens, there has always been a concern about placing these antibiotics topically, as the patient may become sensitized to these drugs. Penicillin allergy is among the highest of all antibiotics and affects about 10% of the population.

The conventional medicament used in root canal treatment is non-setting calcium hydroxide paste. This paste raises the alkalinity and kills many of the bacterial species in endodontic infection. However, the potency of calcium hydroxide against species like Enterococcus faecalis, which is commonly found in failing cases, is diminished by the proton pump and other protective mechanisms. Calcium hydroxide has been shown to inhibit the growth of some species, while in the root canal, but not kill them. Importantly, calcium hydroxide has not been shown to improve the healing following root canal treatment. It is also toxic to host tissues and so has to be confined to the root canal environment and not extruded into the periapical lesion, where some of the bacteria may be present.

Therefore, there is a need for more effective treatments for endodontic infections.

### SUMMARY OF THE INVENTION

One aspect of the invention relates to a method of treating a bacterial infection in a root canal space and/or immediate periapical region of a tooth of a subject in need thereof, the method comprising contacting the root canal space and/or immediate periapical region with a therapeutically effective amount of tigecycline, wherein the contacting comprises topical application of the tigecycline to the root canal space and/or immediate periapical region, thereby treating the bacterial infection in (arising from) the root canal space and/or immediate periapical region of the tooth.

A second aspect of the invention relates to a method of reducing the risk of developing a bacterial infection in a root canal space and/or immediate periapical region of a tooth of a subject in need thereof, the method comprising contacting the root canal space and/or immediate periapical region with a therapeutically effective amount of tigecycline, wherein the contacting comprises topical application of the tigecycline to the root canal space and/or immediate periapical region, thereby reducing the risk of developing a bacterial infection in the root canal space and/or immediate periapical region of the tooth.

These and other aspects of the invention are set forth in more detail in the description of the invention below.

### BRIEF DESCRIPTION OF THE DRAWINGS

FIGS. 1A-1B provides MICs and MBCs of tested antibiotics (FIG. 1A) Minimum Inhibitory Concentration (MIC) and (FIG. 1B) Minimum Bactericidal Concentration (MBC) of Augmentin®, Tigecycline, ciprofloxacin, minocycline and metronidazole measured in ug/mL against F. nucleatum, P. gingivalis, S. intermedius and E. feacalis. *Groups under the same bar are not significantly different. (P<0.05).

FIGS. 2A-2C show comparative analysis of mean color change (ΔE). (FIG. 2A) One week post-treatment; (FIG. 2B) Two-weeks post-treatment and (FIG. 2C) Three-week post-treatment with triple antibiotic paste (TAP), Augmenting (Aug) (i.e., amoxicillin/clavulanate potassium), Tigecycline (Tig) at different concentrations. (P<0.05).

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, 1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control, or a prior value.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control, or a prior value. In particular embodiments, the reduction can result in no or essentially no (i.e., an insignificant amount, e.g., less than about 10% or even 5%) detectable activity or amount.

As used herein, a “root canal space” means the pulp chamber (within the coronal part of the tooth), the main root canal(s), and the branches that may connect the root canals to each other or to the surface of the root. The root canal space is naturally filled with pulp tissue (i.e., dental pulp; soft innermost layer of a tooth that contains blood vessels and nerves). In an endodontic treatment, diseased or damaged pulp tissue is removed from the root canal space and the root canal space filled and sealed. The “main root canal space” is distinct from, but lead to the immediate periapical region, the lateral or accessory canals and dentinal tubules.

As used herein, the term “periapical region” means the area surrounding the apex of the root of a tooth.

As used herein, the term “immediate periapical region” means the periapical region adjacent to the root of a tooth and within about 2 mm to about 3 mm of the root and may include the apical foramen. Thus, in some embodiments, treating the immediate periapical region can mean administering the tigecycline by, for example, introducing a needle about 2 mm through the apical foramen into the immediate periapical region.

A “therapeutically effective” amount as used herein is an amount that provides some improvement or benefit to the subject. Alternatively stated, a “therapeutically effective” amount is an amount that will provide some alleviation, mitigation, or decrease in at least one clinical sign and/or symptom of endodontic disease in the subject. Those skilled in the art will appreciate that the therapeutic effects need not be complete or curative, as long as some benefit is provided to the subject. A benefit may also be found in the rate of healing following treatment, which may be measured by the reduction in size of the periapical lesion with time or the duration needed for radiographic healing of the lesion.

By the terms “treat,” “treating,” or “treatment of,” it is intended that the severity of the subject's condition is reduced or at least partially improved or modified and that some alleviation, mitigation or decrease in at least one clinical symptom is achieved.

“Topical application” as used herein means any method of contacting a root canal space with tigecycline including irrigating the space with a solution comprising tigecycline or otherwise filling the space with tigecycline, for example, as a paste or as incorporated into a hydrogel, a biological agent such as Emdogain® (Straumann USA, Andover, Mass.) (i.e., a gel containing enamel matrix derivative originating from unerupted porcine tooth buds), or other scaffold material, or impregnated into a paper point, each of which is inserted into the canal or otherwise used to medicate the root canal space. Tigecycline may be applied directly as a solution, a paste or a gel, or it may be attached to the surface of microbeads, nanoparticles or quantum particles. Alternatively, tigecycline may be incorporated into root canal core filling material (e.g., gutta percha) or root canal sealers that are used to permanently fill or obturate the root canal space. In some embodiments, tigecycline may be administered via injection into the root canal space into the periapical region and/or into a periapical lesion.

In the context of a topical, therapeutic agent, the application includes extrusion of the material into the region immediately outside the apical foramen (canal terminus), which commonly has an area of inflammation or infection caused by pulpal disease. The surface of the root at the apex and the periapical lesion may become physically permeated by the therapeutic agent used inside the root canal space. The eventual healing of the periapical lesion following endodontic treatment, and the absence of clinical signs and symptoms of disease are the markers of clinical success of treatment.

The present invention is directed to compositions comprising tigecycline and their use in treating endodontic infections and/or inflammation and/or for reducing the risk of developing endodontic infection and/or inflammation, and/or for protecting regenerated pulp tissue from the effects of any residual infection in the root canal.

Tigecycline is a glycylcycline antibiotic derived from the tetracycline family having the structure of:

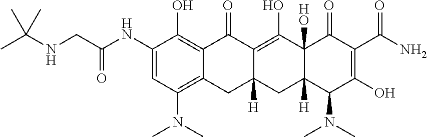 [SMILES: CN(C)c1cc(NC(=O)CNC(C)(C)C)c(O)c2c1C[C@H]1C[C@H]3[C@H](N(C)C)C(O)=C(C(N)=O)C(=O)[C@@]3(O)C(O)=C1C2=O]

Tigecycline (C₂₉H₃₉N₅O₈) is described for intravenous treatment of a variety of bacterial infections. However, as far as inventors are aware, tigecycline has not been described for topical treatment of any disease, in particular, it has not been described for antimicrobial treatment during endodontic procedures.

Thus, in some embodiments, a method of treating a bacterial infection in a root canal space (i.e., pulp space) and/or immediate periapical region of a tooth of a subject in need thereof is provided, the method comprising contacting the root canal space and/or immediate periapical region with a therapeutically effective amount of tigecycline, wherein the contacting comprises topical application of the tigecycline to the root canal space and/or immediate periapical region, thereby treating the bacterial infection in the root canal space and/or immediate periapical region of the tooth.

In some embodiments, a method of reducing the risk of developing a bacterial infection in a root canal space and/or immediate periapical region of a tooth of a subject in need thereof is provided, the method comprising contacting the root canal space and/or immediate periapical region with a therapeutically effective amount of tigecycline, wherein the contacting comprises topical application of the tigecycline to the root canal space and/or immediate periapical region, thereby reducing the risk of developing a bacterial infection in the root canal space and/or immediate periapical region of the tooth.

A subject in need thereof may be any subject having a tooth with partial or complete disease of the pulp tissue present in the root canal space (e.g., pulp space (pulp chamber—within the coronal part of the tooth) and/or in the root canal and/or any lateral or accessory branches, or the immediate periapical region including the immediate periapical region. In addition, avulsed or exarticulated teeth due to trauma may also be treated with this agent by immersion prior to replantation or as a root canal medicament during treatment.

As used herein, “diseased pulp tissue” means pulp tissue that is infected (e.g., a bacterial infection) and/or is damaged (e.g., a traumatic injury).

Thus, a subject may be any mammal having a tooth with diseased pulp tissue. In some embodiments, the subject may be a human having a tooth with diseased pulp tissue.

Teeth with diseased pulp may develop inflammation of the periapical tissue (i.e., structures adjacent to a root apex of a tooth), developing periapical lesions. In an infection setting, bacterial biofilm may be confined to the root canal and bacterial toxins and enzymes may move apically, the bacteria may move into the apical foramen to be located just outside the canal on the external surface of the root, and/or the bacteria may extend directly into the lesion. Thus, in some embodiments, the present invention is directed to a method of reducing inflammation of periapical tissue of a tooth by administering topically into the periapical tissue an effective amount of tigecycline. In some embodiments, the present invention comprises reducing inflammation of periapical tissue of a tooth may be done in combination with treating the root canal space, main root canal space, immediate periapical region and/or periapical region by administering a therapeutically effective amount of tigecycline topically to the root canal space, main root canal space, immediate periapical region and/or periapical lesion. In some embodiments, tigecycline may be applied to the periapical region in conjunction with endodontic surgery, where the gingiva (gum) is reflected, the root apex is sectioned, and the tissue completely or incompletely removed.

Tigecycline placed in the root canal may diffuse into the periapical lesion. However, in addition to this random diffusion effect, tigecycline could also be applied directly (e.g., with a needle) beyond the apical foramen, introducing the tigecycline into the lesion. Thus, in some embodiments, the tigecycline contacted with the root canal space permeates the immediate periapical region. Permeation of the tigecycline into the periapical lesion provides antimicrobial and anti-inflammatory effects on the periapical lesion, and leads to faster rates of healing as compared to a subject not so treated with tigecycline.

In some embodiments, a root canal space and/or immediate periapical region may be contacted with at least one additional therapeutic agent. In some embodiments, the at least one additional therapeutic agent may include, but is not limited to, calcium hydroxide, sodium hypochlorite, chlorhexidine, ethylene diamine tetracetic acid (EDTA), iodine-based formulations, phenolic formulations and/or one or more dental tissue regeneration growth factors. Dental tissue regeneration growth factors may include, but are not limited to, Emdogain®, platelet-derived growth factor (PDGF), bone morphogenic protein (BMP), and/or insulin-like growth factor (IGF).

In some embodiments, the diseased pulp in the tooth of the subject may be in a mature tooth. In some embodiments, the tooth of a subject may be an immature tooth. In the latter cases, pulp regeneration, root maturation and mineralization may be the intent of the treatment in addition to the healing of the periapical lesion.

In some embodiments, contacting of a root canal space and/or immediate periapical region with a therapeutically effective amount of tigecycline begins immediately after an endodontic procedure. An endodontic procedure can be carried out due to an infection of the pulp tissue or due to a traumatic injury to the tooth damaging the pulp tissue. The damaged or infected pulp tissue is removed from the root canal space and the root canal space is then treated to disinfect (kill bacteria) or to prevent or reduce the risk of the occurrence of a bacterial infection. An endodontic procedure may remove part of the diseased pulp (pulpotomy (e.g., the coronal portion of the pulp)) from the subject tooth or the entirety of the diseased pulp (pulpectomy) prior to contacting the root canal space and/or immediate periapical region with a therapeutically effective amount of tigecycline as provided herein. During treatment, the root canal space is irrigated with an antimicrobial, which is typically sodium hypochlorite. Tigecycline may be incorporated into the irrigating solution, as it may bind to dentin and produce a lasting substantive antimicrobial effect. This antimicrobial treatment is done in conjunction with enlargement of the canal space by mechanical instruments to allow adequate permeation of the irrigant and to allow obturation. Alternatively, the tigecycline solution may be used in conjunction with an ultrasonically activated irrigation needle or passive file in the root canal, or atomized and dispersed using ultrasonic energy within the root canal space. The objective of using ultrasonic energy is better penetration and permeation of the tigecycline into all the minute spaces of the root canal space, such as isthmuses, fins, lateral and accessory canals, and possibly the dentinal tubules for maximum disinfection.

The use of tigecycline in root canal irrigation may be done during instrumentation and prior to obturation of the root canal, or initiation of regenerative procedures. This use will rely on the residual effect of the antibiotic on root canal bacteria. The use of tigecycline as a root canal medicament will be for the purpose of disinfecting the root canal for a prolonged period before obturation. This period is typically 1-4 weeks or longer (e.g., 1, 2, 3, 4, 5, 6, 7, 8, or more weeks), as it allows for the material to reach the a more extensive volume of the root canal space, and interfere with the life cycle of both planktonic and biofilm bacteria in the root canal environment.

In some embodiments, the present invention provides a method of treating periodontal (gum) disease (periodontitis) by contacting the teeth and gums of a subject in need thereof with a therapeutically effective amount of tigecycline, wherein the contacting comprises topical application of the tigecycline on the teeth and gums of the subject, thereby treating the periodontal disease.

Tetracyclines are also known to have anti-inflammatory properties, in addition to their antimicrobial properties. This action is thought to be through the reduction of inflammatory mediators, namely matrix metalloproteinases, which are enzymes that cause many of the side effects of inflammation. Therefore, the use of tigecycline may reduce periapical inflammation in sub-antimicrobial concentrations, which may accelerate periapical healing.

In some embodiments, contacting of a root canal space and/or immediate periapical region and/or teeth and gums with a therapeutically effective amount of tigecycline may be for a range of time from about 2 days to about 3 months. Thus, in some embodiments, contacting of a root canal space and/or immediate periapical region with a therapeutically effective amount of tigecycline may be for about 3 days to about 3 months, about 5 days to about 3 months, about a week to about 3 months, about 10 days to about 3 months, about 12 days to about 3 months, about 2 weeks to about 3 months, about 3 weeks to about 3 months, about 4 weeks to about 3 months, about 5 weeks to about 3 months, about 6 weeks to about 3 months, about 7 weeks to about 3 months, about 2 months to about 3 months, about 9 weeks to about 3 months, about 10 weeks to about 3 months, about 11 weeks to about 3 months, about 2 days to about 1 week, about 2 days to about 2 weeks, about 2 days to about 3 weeks, about 2 days to about 4 weeks, about 2 days to about 5 weeks, about 2 days to about 6 weeks, about 2 days to about 7 weeks, about 2 days to about 8 weeks, about 5 days to about 1 week, about 5 days to about 10 days, about 5 days to about 2 weeks, about 5 days to about 3 weeks, about 5 days to about 4 weeks, about 1 week to about 2 weeks, about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 6 weeks to about 7 weeks, about 6 weeks to about 8 weeks, about 6 weeks to about 9 weeks, about 6 weeks to about 10 weeks, about 6 weeks to about 11 weeks, and any range or value therein. In some embodiments, contacting of the root canal space and/or immediate periapical region with a therapeutically effective amount of tigecycline may be for a range of about 1 week to about 4 weeks.

In some embodiments, contacting of a root canal space and/or immediate periapical region and/or teeth and gums with a therapeutically effective amount of tigecycline may be for about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 days, and the like.

In some embodiments, a therapeutically effective amount of tigecycline may be in a range from about 0.1 mg/mL to about 10 g/mL, about 0.1 mg/mL to about 100 mg/mL, or about 0.1 mg/ml to about 10 mg/ml, or any range or value therein. Thus, in some embodiments, a therapeutically effective amount of tigecycline may be about 0.1 mg/mL to about 10 g/mL, about 0.1 mg/mL to about 5 g/mL, about 0.1 mg/mL to about 1 g/mL, about 0.1 mg/mL to about 500 mg/mL, about 0.1 mg/mL to about 100 mg/mL, about 0.1 mg/mL to about 75 mg/mL, about 0.1 mg/mL to about 50 mg/mL, about 0.1 mg/mL to about 25 mg/mL, about 0.1 mg/mL to about 10 mg/mL, about 0.1 mg/mL to about 9 mg/mL, about 0.1 mg/mL to about 8 mg/mL, about 0.1 mg/mL to about 7 mg/mL, about 0.1 mg/mL to about 6 mg/mL, about 0.1 mg/mL to about 5 mg/mL, about 0.1 mg/mL to about 4 mg/mL, about 0.1 mg/mL to about 3 mg/mL, about 0.1 mg/mL to about 2 mg/mL, about 0.1 mg/mL to about 1 mg/mL, about 0.1 mg/mL to about 0.9 mg/mL, about 0.1 mg/mL to about 0.8 mg/mL, about 0.1 mg/mL to about 0.7 mg/mL, about 0.1 mg/mL to about 0.6 mg/mL, about 0.1 mg/mL to about 0.5 mg/mL, about 1 mg/mL to about 10 g/mL, about 1 mg/mL to about 5 g/mL, about 1 mg/mL to about 1 g/mL, about 1 mg/mL to about 500 mg/mL, about 1 mg/mL to about 100 mg/mL, about 1 mg/mL to about 75 mg/mL, about 1 mg/mL to about 50 mg/mL, about 1 mg/mL to about 25 mg/mL, about 1 mg/mL to about 10 mg/mL, about 1 mg/mL to about 5 mg/mL, about 5 mg/mL to about 100 mg/mL, about 5 mg/mL to about 75 mg/mL, about 5 mg/mL to about 50 mg/mL, about 5 mg/mL to about 25 mg/mL, about 5 mg/mL to about 10 mg/mL, about 10 mg/mL to about 100 mg/L, about 10 mg/mL to about 75 mg/mL, about 10 mg/mL to about 50 mg/mL, about 10 mg/mL to about 25 mg/mL, about 20 mg/mL to about 100 mg/mL, about 20 mg/mL to about 90 mg/mL, about 20 mg/mL to about 80 mg/mL, about 20 mg/mL to about 70 mg/mL, about 20 mg/mL to about 60 mg/mL, about 20 mg/mL to about 50 mg/mL, about 20 mg/mL to about 40 mg/mL, about 20 mg/mL to about 30 mg/mL, about 50 mg/mL to about 10 g/mL, about 50 mg/mL to about 5 g/mL, about 50 mg/mL to about 1 g/mL, about 50 mg/mL to about 500 mg/mL, about 50 mg/mL to about 100 mg/mL, about 50 mg/mL to about 75 mg/mL, about 75 mg/mL to about 100 mg/mL, about 100 mg/mL to about 10 g/mL, about 100 mg/mL to about 5 g/mL, about 500 mg/mL to about 10 g/mL, about 500 mg/mL to about 5 g/mL, about 1 g/mL to about 10 g/mL, about 1 g/mL to about 5 g/mL, about 5 g/mL to about 10 g/mL, and any range or value therein. In some embodiments, a therapeutically effective amount of tigecycline may be in a range from about 0.1 mg/mL to about 10 mg/mL or any range or value therein. In some embodiments, a therapeutically effective amount of tigecycline may be in a range from about 0.1 mg/mL to about 1 mg/mL or any range or value therein.

In some embodiments, a therapeutically effective amount of tigecycline may be about 0.1, 0.15, 0.2, 0.25, 0.3, 0.35, 0.4, 0.45, 0.5, 0.55, 0.6, 0.65, 0.7, 0.75, 0.8, 0.85, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 400, 500, 600, 700, 800, 900, 1000, 1500, 2000, 2500, 3000, 3500, 4000, 4500, 5000, 7500, 10,000 mg/mL, and any value therein.

In some embodiments, tigecycline may be administered to the root canal space and/or immediate periapical region of a subject's tooth and/or teeth and gums of a subject one time. In some embodiments, tigecycline may be administered to the root canal space and/or immediate periapical region of a subject's tooth and/or teeth and gums of a subject at least two times (e.g., 2, 3, 4, 5, 6, 7, 8 or more times). Thus, for example, the root canal space of the tooth may be irrigated with a tigecycline solution and then later the tooth may be filled with a filling impregnated with tigecycline. In some cases, the root canal space and/or immediate periapical region of a tooth may require more than one treatment to bring an infection under control. In some embodiments, a tooth may not be filled and permanently sealed until it is completely free of the active infection and/or bacteria. Accordingly, a temporary seal may be placed on the tooth, thereby allowing re-treatment (e.g., contacting 2, 3, 4, 5, or more times) of the root canal space of the tooth with tigecycline until the infection is under control.

Tigecycline may be in any form useful for topical application to a root canal space and/or immediate periapical region and/or teeth and gums. Thus, for example, the tigecycline may be in the form of a solution, a paste, a gel or a slow-release carrier.

In some embodiments, dried/powdered tigecycline may be resuspended in a liquid (e.g., saline) to form a paste.

In some embodiments, a gel or slow-release carrier may comprise a particle (e.g., a nanoparticle, a microparticle, a microbead, nanobead, a polymeric microbead, an alginate microbead) or metallic quantum dot beads. In some embodiments, a gel may be a hydrogel, e.g., an alginate-fibrinogen hydrogel, or an oxidized alginate hydrogel particle (e.g., microbead) or combined with ethylene glycol, polylactic acid, or a biological agent such as Emdogain® (Straumann USA, Andover, Mass.). In some embodiments, the gel or a slow-release carrier is biodegradable. The slow release of the antibiotic may be at a rate that produces an effective antimicrobial dose over about 1 to about 90 days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days, and range or value therein) (e.g., about 1 to about 7 days, about 1 to about 10 days, about 1 to about 14 days, about 1 to about 20 days, about 1 to about 24 days, about 1 to about 45 days, about 1 to about 60 days, about 7 to about 14 days, about 7 to about 20 days, about 7 to about 24 days, about 7 to about 45 days, about 7 to about 60 days, about 14 to about 20 days, about 14 to about 24 days, about 14 to about 45 days, about 14 to about 60 days, about 14 to about 90 days, about 21 to about 24 days, about 21 to about 45 days, about 21 to about 60 days, about 21 to about 90 days, about 24 days to about 45 days, about 24 to about 60 days, about 24 to about 90 days, about 36 to about 45 days, about 36 to about 60 days, about 36 to about 90 days, about 45 to about 60 days, about 45 to about 60 days, about 45 to about 90 days, about 60 to about 90 days, and any range or value therein).

In some embodiments, the tigecycline may be impregnated in material useful for disinfecting a root canal. Thus, for example, tigecycline may be impregnated into a filling material, a paper point or a scaffold material. In some embodiments, the scaffold material may be polylactic acid (PLA) (e.g., poly-L-lactic acid), polyglycolic acid (PGA), polyglycolic acid-poly-L-lactic acid (PGA-PLLA), polylactic polyglycolic acid (PLGA), chitosan, collagen, alginate, hyaluronic acid, dentin, Emdogain® (i.e., a gel containing enamel matrix derivative originating from unerupted porcine tooth buds) incorporated in products such as GELFOAM® (absorbable gelatin powder), COLLACOTETM/COLLAPLUG® (absorbable collagen wound dressings), silk, VICRYL® (polyglactin 910), chromic catgut and/or other suture materials, and the like. In some embodiments, tigecycline may be electrospun into a polymer of the scaffold. In some embodiments, a tigecycline impregnated scaffold may release tigecycline over time as it degrades. In some embodiments, a tigecycline impregnated scaffold may be biodegradable.

In some embodiments, tigecycline may be provided in a composition comprising growth factors. In some embodiments, tigecycline maybe combined with an enamel matrix derivative, e.g., Emdogain®, a commercially available growth factor formulation for regeneration of dental tissues. In some embodiments, tigecycline may be provided in a composition comprising at least one of platelet-derived growth factor (PDGF), bone morphogenic protein (BMP), and/or insulin-like growth factor (IGF). In some embodiments, additional growth factors may be included in a composition comprising tigecylcine and Emdogain®. The combination of tigecycline and at least one growth factor offers an easily applied mixture in the root canal system, given the viscosity and deliverability of enamel matrix derivative gels (e.g., Emdogain gel). Without being bound to any particular theory, it is believed that an antimicrobial/growth factor mixture may be more efficacious in regenerative endodontic therapy than applying each agent on its own.

In some embodiments, a filling material for root canal space may be a thermoplastic material. Thus, in some embodiments, a thermoplastic material may be gutta-percha (a rigid natural latex (e.g., a polyterpene, a polymer of isoprene, or polyisoprene (e.g., trans-1, 4-polyisoprene) from Palaquium spp. (e.g. Palaquium gutta)). In some embodiments, the filling may be a synthetic polyester polymer (e.g., Resilon®). In some embodiments the filling material may be incorporated in a root canal sealer that can be used to permanently fill the root canal. Root canal sealers may include, but are not limited to, sealers comprising a zinc oxide and eugenol base, a epoxy resin base, a calcium hydroxide-base, a tricalcium silicate base or a bioceramic base. In some embodiments, a root canal sealer may further comprise barium sulfate, a radio-opacifier. In some embodiments, a filling material useful with this invention may be impregnated with tigecycline.

In some embodiments, the root canal space may be irrigated following treatment to remove the tigecycline before the tooth is filled and permanently sealed or between treatments with tigecycline.

Tigecycline is effective as an antibiotic against a broad spectrum of endodontic or oral bacteria. In some embodiments, tigecycline is effective against a gram-positive and/or gram-negative bacteria. Non-limiting examples of bacteria for which tigecycline is effective may include members of the following bacterial genera: Enterococcus, Streptococcus, Peptostreptococcus, Parvimonas, Staphylococcus, Lactobacillus, Eubacterium, Actinomyces, Eikenella, Corynebacterium, Clostridium, Filifactor, Atopobium, Rothia, Burkholderia, Olsenella, Gemella, Porphyromonas, Prevotella, Tanerella, Treponema, Fusobacterium, Aggregatibacter, and/or Bacteroides,

In addition to being effective on a broad range of endodontic pathogens, the inventors have found that tigecycline may provide less tooth discoloration (e.g., crown discoloration) than other antibiotics used in endodontic procedures (at the same concentrations). Crown discoloration was reported in 40% of the studies on regenerative endodontic treatments (8). Such complications in anterior teeth can affect a patient's quality of life (9). For example, minocycline in TAP causes severe tooth discoloration (10). Thus, in some embodiments, at concentrations of about 0.1 mg/mL to about 1 mg/mL, tigecycline not only reduces bacterial growth but also results in less tooth discoloration as compared to standard endodontic antibiotics (e.g., TAP). In addition, at concentrations greater than 1 mg/ml to about 10 mg/mL and after applying an inner bonding within the coronal endodontic access preparation, no detectable discoloration was observed with tigecycline. Further, tigecycline falls into the group of antibiotics generally considered to have low allergenicity as compared to, for example, amoxicillin, penicillin and other β-lactam type antibiotics. These attributes of high efficacy at low concentrations (high concentrations of antibiotics are toxic to the stem cells of apical papilla (11, 12)), low tooth discoloration and low allergenicity make tigecycline an excellent candidate for impregnation into scaffolds for use in tissue development.

While diseased dental pulp is traditionally removed and replaced with materials such as gutta-percha and root canal sealer, in some cases, it may be possible to regenerate pulp or pulp-like tissue into the root canal space, thereby replacing the diseased pulp-tissue with regenerated tissues. Disinfection of the root canal space and immediate periapical region and maintaining a disinfected space is essential for tissue regeneration. Thus, in some embodiments, the methods of the invention may be used to disinfect the root canal space and immediate periapical region prior to tissue regeneration. Tigecycline may provide broad spectrum antibiotic effect at concentrations that are non-toxic to cells for tissue regeneration and which have reduced tooth discoloration.

Accordingly, in some embodiments, the invention provides a method of regenerating tissue in a root canal space, the method comprising contacting the root canal space with a therapeutically effective amount of tigecycline (as described herein); and contacting the root canal space with cells (e.g., stem cells, hematopoietic cells), thereby regenerating pulp tissue.

In some embodiments, the root canal space may be contacted with the tigecycline concurrently (at the same time) with the cells. In some embodiments, the root canal space may be contacted with the tigecycline prior to being contacted with the cells. In some embodiments, the root canal space may be contacted with the tigecycline after being contacted with the cells. In some embodiments, the root canal space may be contacted with the tigecycline prior to and concurrently with being contacted with the cells. In some embodiments, the root canal space may be contacted with the tigecycline prior to, concurrently with and after being contacted with the cells.

Scaffolds may be used in the process of regenerating pulp tissue. In some embodiments, stem cells and the like are seeded onto a scaffold for regeneration of pulp tissue in the root canal space. In some embodiments, the tigecycline may be impregnated in material useful in methods for regeneration of pulp tissue. Tigecycline may be impregnated into a scaffold material to maintain a disinfected space during the regeneration process. In some embodiments, tigecycline is impregnated into a scaffold at low concentrations such as, for example, 0.1 mg/mL to about 1 mg/mL, or about 0.1 mg/mL to about 10 mg/mL as described herein.

In some embodiments, a scaffold material useful with this invention can include, but is not limited to, polylactic acid (PLA) (e.g., poly-L-lactic acid-based (PLLA)), polyglycolic acid (PGA), polyglycolic acid-poly-L-lactic acid (PGA-PLLA), polylactic polyglycolic acid (PLGA), chitosan, collagen, alginate, hyaluronic acid, dentin, and/or silk, and the like. In some embodiments, the scaffold may be a nanofibrous scaffold. In some embodiments, the scaffold may be a nanofibrous scaffold comprising PLLA.

The tigecycline may be electrospun into a polymer of a scaffold.

In some embodiments, a tigecycline impregnated scaffold may provide a slow release of the tigecycline over time as the scaffold degrades. The rate of tigecycline release at an effective antimicrobial, and possibly anti-inflammatory dose may be over 1-90 days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days, and range or value therein) (e.g., about 1 to about 7 days, about 1 to about 10 days, about 1 to about 14 days, about 1 to about 20 days, about 1 to about 24 days, about 1 to about 45 days, about 1 to about 60 days, about 7 to about 14 days, about 7 to about 20 days, about 7 to about 24 days, about 7 to about 45 days, about 7 to about 60 days, about 14 to about 20 days, about 14 to about 24 days, about 14 to about 45 days, about 14 to about 60 days, about 14 to about 90 days, about 21 to about 24 days, about 21 to about 45 days, about 21 to about 60 days, about 21 to about 90 days, about 24 days to about 45 days, about 24 to about 60 days, about 24 to about 90 days, about 36 to about 45 days, about 36 to about 60 days, about 36 to about 90 days, about 45 to about 60 days, about 45 to about 60 days, about 45 to about 90 days, about 60 to about 90 days, and any range or value therein).

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

### Study of Tooth Discoloration

### Bacterial Strain and Media

Four common endodontic pathogens were selected for the study: Enterococcus faecalis (ATCC 4083), Streptococcus intermedius (ATCC 31412), Fusobacterium nucleatum (ATCC 49256), and Porphyromonas gingivalis (ATCC 33277). All four bacterial strains were initially grown on anaerobic blood agar plates in the anaerobic chamber (Mini-MACS, Microbiology international, USA) in 10% CO2, 5% H2, and 85% N2 at 37 ° C. Then, each microorganism was cultured in their recommended broth as follow; brain heart infusion (BHI) broth for E. feacalis, Todd Hewitt broth for S. intermedius, chopped meat for F. nucleatum, and tryptic soy broth supplemented with hemin and vitamin K for P. gingivalis. All bacteria were grown to turbidity of 1.0 McFarland density (3×10⁶ CFU/mL), determined using a spectrophotometer.

### Minimum Bactericidal Concentration (MBC) and Minimum Inhibitory Concentration (MIC)

MIC and MBC of metronidazole, ciprofloxacin, minocycline, Augmentin®, tigecycline were determined against the four study bacteria using E-test (Etest®, bioMérieux, USA) as previously described (13).

### Bacteria Inoculation in Root Canals

One hundred and twenty intact single rooted permanent human teeth were collected for the study. All teeth were autoclaved, then access cavities were prepeared and the canal instrumented to size 40/0.04, with saline irrigation. Teeth were autoclaved again to ensure absence of bacteria prior to bacterial inoculation. The root canals were then inoculated with the bacterial mixture, using syringe delivery, with the 30-gauge needle close to the working length. Access cavities were then sealed with a sterile sponge and Cavit (3M ESPE). All teeth were then incubated in the anaerobic chamber. The suspensions were replenished weekly for 3 weeks to allow for biofilm formation and maturation (14).

### Preparation of Hydrogel as a Scaffold for Antibiotics

The tested antibiotics at concentrations of 0.1 mg/ml and 1.0 mg/ml solutions were prepared and mixed with oxidized alginate solution to prepare the hydrogel microbeads, as previously described (15). Briefly, sodium alginate was dissolved in distilled water to make a 1% solution. Then 1.51 mL of 0.25 M sodium periodate was added in 100 ml alginate solution, with continuous stirring, to react in dark at room temperature for 24 h. The oxidizing reaction was stopped by adding 1 g ethylene glycol and then 2.5 g sodium chloride was added in the solution. An excess amount of ethanol (200 mL) was added to precipitate the product, which was then collected by centrifuging. The precipitates were re-dissolved in 100 mL distilled water and precipitated again with 200 mL of ethanol. The second precipitates were collected and dissolved in 30 mL distilled water. The final product was obtained through freeze-drying under reduced pressure for 24 hours.

Initially, a 12 mg/mL solution of 7.5% oxidized alginate in 0.9% sodium chloride was prepared. Then fibrinogen from bovine plasma was added in at the concentration of 1 mg/mL and incubated at 37° C. from 1 to 2 hours to make a mixed solution of oxidized alginate-fibrinogen. A 100 mmol/L calcium chloride solution in distilled water was prepared and thrombin was then added in at the concentration of 1 unit/mL to make a mixed crosslinking solution. The two kinds of solution were sterilized separately by filtering through 0.2 μn hydrophilic PTFE membrane. Hydrogel bead formation was accomplished by extruding oxidized alginate-fibrinogen solution droplets into the mixed crosslinking solution of calcium chloride and thrombin.

Antibiotic solutions were prepared and suspended in the oxidized alginate-fibrinogen solution. The suspension was then loaded into a syringe, which was placed into a syringe pump and connected to a bead-generating device (Var J1, Nisco, Zurich, Switzerland). Nitrogen gas was fed to the gas inlet and a pressure of 8 psi was established to form a coaxial airflow to break up the alginate-fibrinogen-cell droplets. The CaCl₂-thrombin solution was filled in a wide plate to collect the droplets and simultaneously stirred by a magnetic bar. This produced small, oxidized alginate-fibrin-antibiotics beads.

Root Canal Dressing with Antibiotics/Hydrogel Scaffolds:
Root canals (n=15 per group) were filled randomly with antibiotics in hydrogel scaffolds as follow:
   Group 1: no antibiotics (negative control),
   Group 2: Augmentin® (0.1 mg/mL),
   Group 3: Tigecycline (0.1 mg/mL),
   Group 4: TAP (0.1 mg/mL),
   Group 5: Augmentin® (1 mg/mL),
   Group 6: Tigecycline (1 mg/mL)
   Group 7: TAP (1 mg/mL),
   Group 8: TAP (1 g/mL) paste mixed with normal saline not the scaffold (positive control).
Next, access cavities were sealed with sterile sponge and Cavit (3M ESPE) and incubated anaerobically for 1 week.

### Post-Treatment Microbial Sampling

One week following antibiotics dressing, root canals were irrigated with 20 mL sterile saline to the working length for 10 minutes to remove the canal contents. Root canal walls were then instrumented with size 40 K file to disrupt any residual biofilms and were transferred to a vial containing normal saline for sampling. Size 2 Gates Glidden burs were used in the root canals dentin followed by three consecutive sterile paper points, which were transferred to the same vial for sampling. The samples were then serially diluted and plated on anaerobic blood agar plates and CFUs were enumerated. Log 10 CFUs were used for statistical analysis.

### Color Change Measurement

Crown color was measured as described previously (16). All color measurements were taken in a dark room inside the anaerobic chamber. The instrument was calibrated before the measurement in each group.
The mean baseline color measurements were taken prior to filling the root canal with the antibiotics (C0) and then at 1 week (C1), 2 weeks (C2), and 3 weeks (C3). The results were expressed in the CIE L*a*b* system (L* [black-white], a* [red-green] and b* [blue-yellow]).
Mean color change (ΔE) value was calculated using the following formula; (ΔE)=[(ΔL*)²+(Δa*)²+(Δb*)²].

### Statistical Analysis

One-way analysis of variance (ANOVA), followed by the Tukey's HSD (with p<0.05) was used for the four microorganisms to compare: A) the MICs and MBCs among tested antibiotics, B) the difference between baseline CFUs and post-treatment CFUs for different antibiotics/scaffold formulations and, C) the changes in color among the groups. Chi square test was used to compare the number of samples that had bacterial growth with those with no bacterial growth for each antibiotic separately.

### Example 2

### Results

Minimum Bactericidal Concentration (MBC) and Minimum Inhibitory Concentration (MIC)

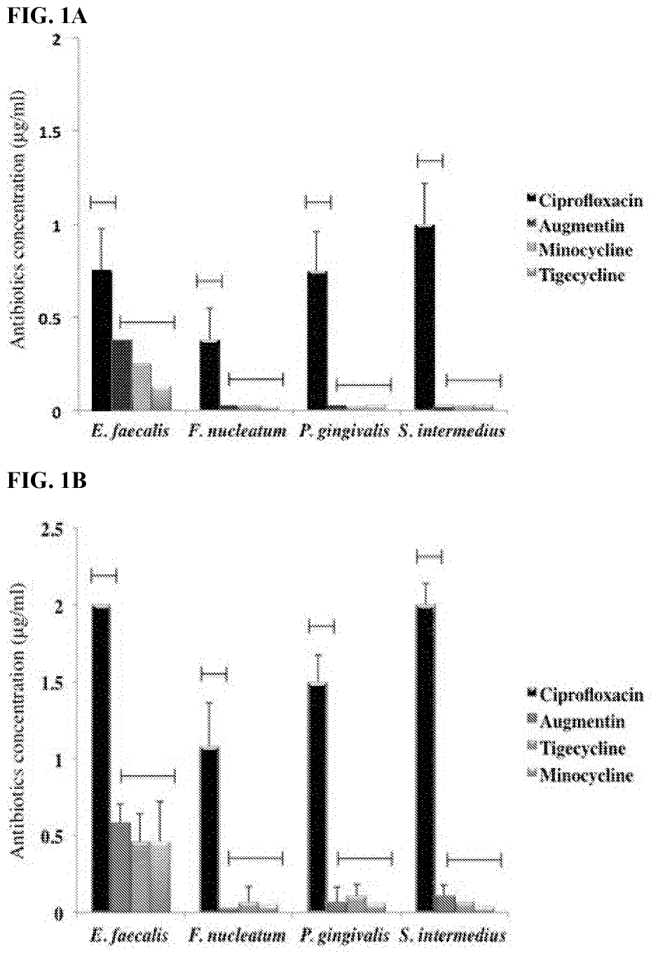

The mean MICs and MBCs of tested antibiotics are shown in FIGS. 1A-1B. Ciprofloxacin had significantly higher MIC and MBC compared to other tested antibiotics. (P<0.05). In addition, metronidazole was not efficacious and had no inhibition zone against any of tested microorganisms

### Colony Forming Units (CFUs)

The results of CFUs are summarized in Table 1. All samples from the control group showed a bacterial growth (mean CFUs 1.3×10⁸ cells/mL). TAP, Augmentin®, and tigecycline at 0.1 mg/mL concentration resulted in 3-4 folds log reduction of CFUs. In addition, TAP, Augmentin®, and tigecycline at 1 mg/mL concentration resulted in 6-7 folds log reduction of CFUs. These differences were statistically significant (P<0.05). TAP at 1 g/mL concentration group resulted in no bacterial growth. There were no significant differences in CFUs between 0.1 mg/mL of TAP, Tigecycline, and Augmentin® (Table 1). There were no significant differences between 1 mg/mL of TAP, Tigecycline, Augmentin® and TAP (Table 1).

TABLE 1
Results of one-way ANOVA comparing
log10 CFUs of the antibiotics groups.

| Group | Log10 | SD | F | P value |
|---|---|---|---|---|
| Control group | 8.13 ^{a} | 0.95 | 71.295 | 0.0005 |
| TAP (0.1 mg/ml) | 4.24 ^{b} | 0.82 | | |
| Tigecycline (0.1 mg/ml) | 3.38 ^{b} | 1.33 | | |
| Augmentin (0.1 mg/ml) | 3.96 ^{b} | 1.02 | | |
| TAP (1 mg/ml) | 0.71 ^{c} | 1.22 | | |
| Tigecycline (1 mg/ml) | 0.93 ^{c} | 1.38 | | |
| Augmentin (1 mg/ml) | 1.06 ^{c} | 1.35 | | |
| TAP (1 g/ml) | 0.00 ^{c} | 0.00 | | |

Number of Samples with Growth

All samples in the control group had bacterial growth (100%). TAP at high concentration (1 g/mL) had the highest effectiveness among tested antibiotics with no bacterial growth in any sample. Chi square analyses showed significantly greater percentage of samples with growth at 0.1 mg/mL as compared to 1 mg/mL for all three antibiotics (p<0.05). TAP, tigecycline and Augmentin® at 1 mg/mL had bacterial growth in 26.7%, 33.3% and 40% of the samples, respectively. TAP, tigecycline and Augmentin® at 0.1 mg/mL had bacterial growth in 100%, 93.3% and 93.3% of the samples, respectively. The differences between 1 mg/mL and 0.1 mg/mL groups were significant (P<0.05). Recognizing that antimicrobial effectiveness represents a spectrum of activities, the higher the dose would be more effective, but the lower dose of 0.1 mg/mL was also significantly more effective than controls.

Discoloration by Antibiotics

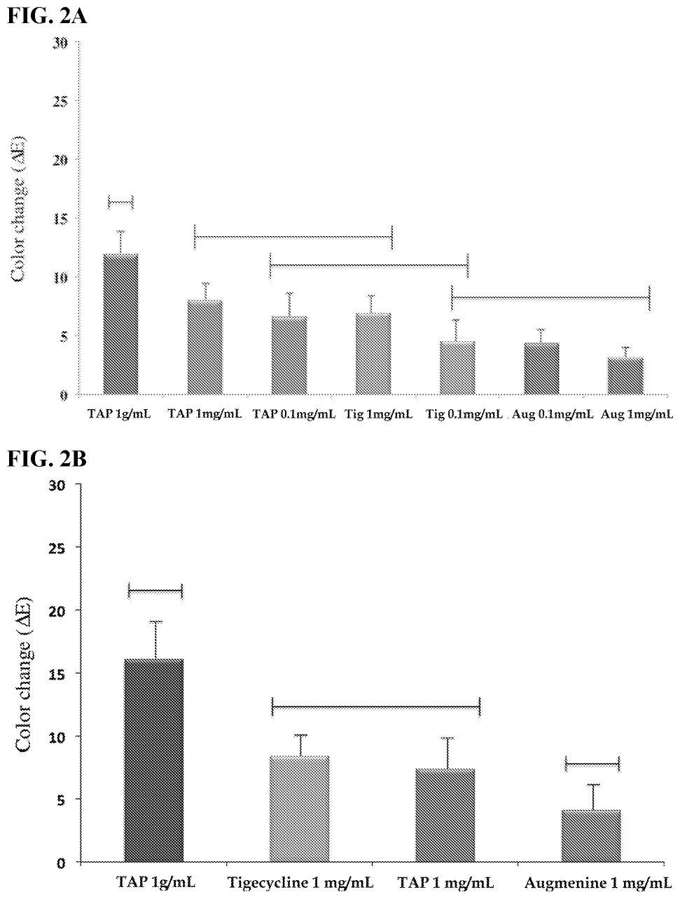

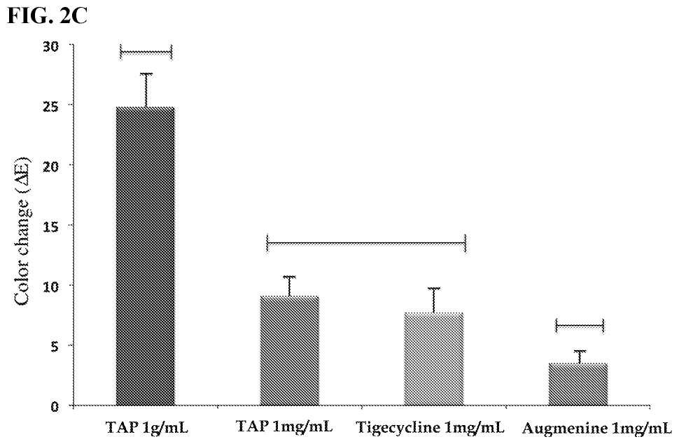

Color change values for all experimental groups are presented in FIGS. 2A-2C. At one week, teeth treated with Augmentin® at 1 mg/mL concentration had significantly lower color change than the other four experimental groups (ΔE=3.1±1.1) followed by tigecycline and TAP at 1 mg/ml (ΔE=6.9±1.9 and 8.0±1.7), respectively (P<0.05). On the other hand, teeth treated with TAP at 1 g/ml concentration had significantly higher color change than other four experimental groups (ΔE=11.9±2.4) (P<0.05) (FIG. 2A).

At 2 and 3 weeks post-treatment, Augmentin® at 1 mg/ml concentration had significantly lower color change (ΔE=4.1±1.9) followed by TAP and Tigecycline at 1 mg/ml (ΔE=7.4±1.9 and 8.4±2.0), respectively (P<0.05). TAP at 1 g/ml concentration had significantly higher color change at 2 weeks (ΔE=16.1±2.7) and 3 weeks (ΔE=24.7±2.7) compared to other four experimental groups at the same time (P<0.05) (FIGS. 2B-2C).

### Example 3

### Discussion

The ex vivo study provided in Examples 1 and 2 evaluated the relative antibacterial efficacy and discoloration potential of several antibiotics and concentrations used as intra-canal medicaments, using a polymicrobial bacterial model. Infected root canal spaces have a highly diverse microflora (17). However, the majority of ex vivo studies used only E. faecalis as a single bacteria target. To avoid this limitation, we tested the antibacterial efficacy against a biofilm composed of four common endodontic microorganisms, including strict gram-negative anaerobes and facultative gram-positive bacteria in an anaerobic environment. We followed the biofilm maturation schedule reported by Stojicic et al (18) for a single organism, and reported on the outcome with any of the microorganisms or their combinations, and not a specific species. This appeared to be a reasonable approach, since the control group had bacterial growth at CFUs of 1.3×10⁸.

Three antibiotics and antibiotic combinations were selected in this study. TAP is the most commonly used antibiotic for regenerative endodontic procedure (4, 7). Augmentin®, which is a combination of amoxicillin and clavulanic acid was found to be effective against 100% of endodontic bacteria (13, 19), and was successfully used in regenerative endodontic treatment (20). Tigecycline is a newer member of tetracyclines, which was also shown to be highly effective against bacteria from necrotic root canals (19). Our findings showed that antibiotics were equivalent in their efficacy at the same concentrations, and that 1 mg/mL was significantly more efficacious compared to 0.1 mg/mL. Previous data indicated that 1 mg/mL concentration of antibiotics may also be biocompatible (11, 12).

Low concentrations of antibiotics in saline results in a watery consistency. This is difficult to deliver in the root canal space as intracanal medicament and would not be suitable as a scaffold. In our study, a previously validated, biocompatible and biodegradable hydrogel scaffold (15) was used to facilitate the delivery of low concentrations (0.1 and 1 mg/mL) antibiotics in the root canal space. Importantly, this approach retained the antimicrobial properties of the antibiotics.

Previously, 0.125 mg/mL of TAP and DAP (metronidazole and ciprofloxacin) had a significant antibacterial effect on E. faecalis biofilm compared to positive control group (no antibiotics), and TAP at 1 mg/mL had significantly lower CFUs compared to TAP at 0.1 mg/mL (21). Additionally, DAP at 500 mg/mL had a significantly higher anti-biofilm (complete eradication) of E. faecalis compared with DAP at 1 mg/mL and 0.1 mg/mL concentrations (22). Our findings generally corroborate these findings, and expand them to other antibiotics and to a polymicrobial biofilm

This research is the first report of the coronal discoloration induced by various intra-canal antibiotic pastes at different concentrations. Our results showed a dose-dependent effect of antibiotics on teeth discoloration. The highest level of coronal discoloration occurred in teeth filled with TAP at 1, 2 and 3 weeks, and the degree of discoloration induced in teeth medicated with 1 g/mL TAP increased overtime. Our results are consistent with previous studies that evaluated crown discoloration induced by antibiotic pastes (16, 23-25). TAP discolors teeth significantly more than DAP (23). Minocycline is an effective antibiotic against endodontic bacteria (26). Our study showed significantly lower MIC and MBC for minocycline compared to ciprofloxacin.

Within the limitation of this ex vivo study, TAP at high concentration (1 g/mL) was the most efficacious antibiotic against common endodontic bacterial biofilms but caused the greatest tooth discoloration. On the other hand, TAP, Augmentin® and tigecycline at low concentrations (1 mg/mL) reduced bacterial growth significantly with a minimum color change when applied for one week. The discoloration effect was concentration-dependent except for Augmentin®, which caused minimal color change values at 0.1 and 1 mg/mL concentrations. Clinical studies are needed to confirm these ex vivo findings.

### REFERENCES

1. Verma P, Nosrat A, Kim J R, Price J B, Wang P, Bair E, et al. The Effect of Residual Bacteria on the Outcome of Pulp Regeneration in-vivo. J Dent Res 2017;96:100-6.
2. Fouad A F. The microbial challenge to pulp regeneration. Advances in dental research 2011;23:285-9.
3. Fouad A, Nosrat A. Pulp regeneration in previously infected root canal space. Endodontic Topics 2013;28:24-37.
4. Banchs F, Trope M. Revascularization of immature permanent teeth with apical periodontitis: new treatment protocol? J Endod 2004;30:196-200.
5. Nosrat A, Seifi A, Asgary S. Regenerative endodontic treatment (revascularization) for necrotic immature permanent molars: a review and report of two cases with a new biomaterial. J Endod 2011;37:562-7.
6. Nosrat A, Homayounfar N, Oloomi K. Drawbacks and unfavorable outcomes of regenerative endodontic treatments of necrotic immature teeth: a literature review and report of a case. J Endod 2012;38:1428-34.
7. Hoshino E, Kurihara-Ando N, Sato I, Uematsu H, Sato M, Kota K, et al. In-vitro antibacterial susceptibility of bacteria taken from infected root dentine to a mixture of ciprofloxacin, metronidazole and minocycline. International endodontic journal 1996;29:125-30.
8. Torabinejad M, Nosrat A, Verma P, Udochukwu O. Regenerative Endodontic Treatment or Mineral Trioxide Aggregate Apical Plug in Teeth with Necrotic Pulps and Open Apices: A Systematic Review and Meta-analysis. J Endod 2017.
9. Kahler B, Rossi-Fedele G. A Review of Tooth Discoloration after Regenerative Endodontic Therapy. J Endod 2016;42:563-9.
10. Kim J H, Kim Y, Shin S J, Park J W, Jung W. Tooth discoloration of immature permanent incisor associated with triple antibiotic therapy: a case report. J Endod 2010;36:1086-91.
11. Ruparel N B, Teixeira F B, Ferraz C C, Diogenes A. Direct effect of intracanal medicaments on survival of stem cells of the apical papilla. Journal of endodontics 2012;38:1372-5.
12. Althumairy R I, Teixeira F B, Diogenes A. Effect of dentin conditioning with intracanal medicaments on survival of stem cells of apical papilla. Journal of endodontics 2014;40:521-5.
13. Baumgartner J C, Xia T. Antibiotic susceptibility of bacteria associated with endodontic abscesses. Journal of endodontics 2003;29:44-7.
14. Stojicic S, Shen Y, Haapasalo M. Effect of the source of biofilm bacteria, level of biofilm maturation, and type of disinfecting agent on the susceptibility of biofilm bacteria to antibacterial agents. Journal of endodontics 2013;39:473-7.
15. Zhou H, Xu H H. The fast release of stem cells from alginate-fibrin microbeads in injectable scaffolds for bone tissue engineering. Biomaterials 2011;32:7503-13.
16. Lenherr P, Allgayer N, Weiger R, Filippi A, Attin T, Krastl G. Tooth discoloration induced by endodontic materials: a laboratory study. International endodontic journal 2012;45:942-9.
17. Li L, Hsiao W W, Nandakumar R, Barbuto S M, Mongodin E F, Paster B J, et al. Analyzing endodontic infections by deep coverage pyrosequencing. J Dent Res 2010;89:980-4.
18. Stojicic S, Shen Y, Haapasalo M. Effect of the source of biofilm bacteria, level of biofilm maturation, and type of disinfecting agent on the susceptibility of biofilm bacteria to antibacterial agents. Journal of endodontics 2013;39:473-7.
19. Jungermann G B, Burns K, Nandakumar R, Tolba M, Venezia R A, Fouad A F. Antibiotic resistance in primary and persistent endodontic infections. Journal of endodontics 2011;37:1337-44.
20. Nosrat A, Li K L, Vir K, Hicks M L, Fouad A F. Is pulp regeneration necessary for root maturation? J Endod 2013;39:1291-5.
21. Sabrah A H, Yassen G H, Liu W C, Goebel W S, Gregory R L, Platt J A. The effect of diluted triple and double antibiotic pastes on dental pulp stem cells and established Enterococcus faecalis biofilm. Clinical oral investigations 2015;19:2059-66.
22. Tagelsir A, Yassen G H, Gomez G F, Gregory R L. Effect of Antimicrobials Used in Regenerative Endodontic Procedures on 3-week-old Enterococcus faecalis Biofilm. Journal of endodontics 2015.
23. Akcay M, Arslan H, Yasa B, Kavrik F, Yasa E. Spectrophotometric analysis of crown discoloration induced by various antibiotic pastes used in revascularization. Journal of endodontics 2014;40:845-8.
24. Kohli M R, Yamaguchi M, Setzer F C, Karabucak B. Spectrophotometric Analysis of Coronal Tooth Discoloration Induced by Various Bioceramic Cements and Other Endodontic Materials. Journal of endodontics 2015;41:1862-6.
25. Nagata J Y, Gomes B P, Rocha Lima T F, Murakami L S, de Faria D E, Campos G R, et al. Traumatized immature teeth treated with 2 protocols of pulp revascularization. J Endod 2014;40:606-12.
26. Jaouni M, Tolba M, Romberg E, Nemieboka N, Fouad A F. Penetration of systemic antibiotics into necrotic pulp space. Journal of endodontics 2010;36:558.

The foregoing is illustrative of the present invention, and is not to be construed as limiting thereof. The invention is defined by the following claims, with equivalents of the claims to be included therein.

1. A method of treating and/or reducing the risk of developing a bacterial infection in a root canal space and/or immediate periapical region of a tooth of a subject in need thereof, comprising
contacting the root canal space and/or immediate periapical region with a therapeutically effective amount of tigecycline, wherein the contacting comprises topical application of the tigecycline to the root canal space, thereby treating and/or reducing the risk of developing the bacterial infection in the root canal space and/or immediate periapical region of the tooth.

2. (canceled)

3. The method of claim 1, further comprising reducing inflammation of periapical tissue of the tooth by contacting the periapical tissue with the therapeutically effective amount of tigecycline.

4. The method of claim 1, further comprising contacting the root canal space and/or immediate periapical region with at least one additional therapeutic agent.

5. The method of claim 4, wherein the at least one additional therapeutic agent is calcium hydroxide, sodium hypochlorite, chlorhexidine and/or dental tissue regeneration growth factors.

6. The method of claim 1, wherein the tooth of the subject is a mature tooth.

7. The method of claim 1, wherein the tooth of the subject is an immature tooth.

8. The method of claim 1, wherein the therapeutically effective amount of tigecycline is about 0.01 mg/mL to about 100 mg/mL.

9. The method of claim 8, wherein the therapeutically effective amount of tigecycline is about 0.1 mg/mL to about 10 mg/mL.

10. The method of claim 1, wherein the contacting of the root canal space with the therapeutically effective amount of tigecycline is for about two days to about three months, optionally for about one week to about four weeks.

11. (canceled)

12. The method of claim 10, further comprising irrigating the root canal space to remove the tigecycline.

13. The method of claim 1, wherein the tigecycline is in the form of a paste, a gel or a slow-release carrier.

14. The method of claim 13, wherein the gel or slow-release carrier is in a microparticle form.

15. The method of claim 13, wherein the gel is a hydrogel.

16. The method of claim 13, wherein the gel is an alginate-fibrinogen hydrogel.

17. The method of claim 13, wherein the gel is an oxidized alginate hydrogel microbead.

18. The method of claim 1, wherein the tigecycline is impregnated into a filing material, a paper point or a scaffold material.

19. The method of claim 18, wherein the scaffold material is polylactic acid (PLA), polyglycolic acid (PGA), polyglycolic acid-poly-L-lactic acid (PGA-PLLA), polylactic polyglycolic acid (PLGA), chitosan, collagen, alginate, hyaluronic acid, dentin, silk and/or EMDOGAIN®.

20. The method of claim 18, wherein the root canal filling material is a thermoplastic material or is a root canal sealer that fills the root canal permanently.

21. The method of claim 20, wherein the thermoplastic material is gutta-percha.

22. The method of claim 1, wherein the tigecycline contacted with the root canal space permeates the immediate periapical region providing antimicrobial and anti-inflammatory effects on a periapical lesion, and leading to faster rates of healing.
